(19) United States
(12) Patent Application Publication
(10) Pub. No.: US 2025/0107705 A1
(43) Pub. Date: Apr. 3, 2025

(54) MEDICAL DEVICE FOR THE EAR CANAL

(71) Applicant: TYMPANY MEDICAL LIMITED, Galway (IE)

(72) Inventors: Rory O'Callaghan, Co. Cavan (IE); Elizabeth McGloughlin, Limerick (IE); Christina Walsh, Galway (IE); Neil Clancy, Kildare (IE); Mark Butler, Ennis (IE); Ian Walton, Co. Mayo (IE)

(21) Appl. No.: 18/729,970

(22) PCT Filed: Jan. 24, 2023
(86) PCT No.: PCT/IB2023/000036

§ 371 Date: Jul. 18, 2024

Related U.S. Application Data
(60) Provisional application No. 63/302,470, filed on Jan. 24, 2022.

(51) Int. Cl. A61B 1/227; A61B 1/00; A61B 1/05; A61B 1/07; A61B 1/32

(52) U.S. Cl. CPC A61B 1/227; A61B 1/00147; A61B 1/05; A61B 1/07; A61B 1/32

(57) ABSTRACT

The invention provides a medical device, kit, system, and method that may be used for viewing the ear during surgery on a subject. The invention may allow a surgeon to view the surgical site with a camera mounted on a probe and maintain patency of the ear canal, while keeping both hands free for surgical techniques.

### FIELD OF THE INVENTION

The present invention relates to a surgical device and kits, systems, and methods of use thereof, including but not limited to surgical treatments of the ear.

### BACKGROUND

Traditional ear surgery uses microscopic visualization with a dynamic bi-manual technique. The major disadvantage of this approach is the narrow field of view looking down into the ear canal. This can lead to poor visualization of disease in difficult-to-access areas in the middle ear, such as the sinus tympani and facial recess. Because traditional microscopy provides a narrow field of view, its use in the ear necessitates exposing the middle ear and attic area by performing a mastoidectomy, a surgical procedure which opens the mastoid bone. Mastoidectomy procedures are associated with increased operating times, more serious complications, longer hospital stays, and protracted recovery for patients.

More recently, a single-handed endoscope assisted technique has gained popularity. Although views are far superior, using one hand to perform middle ear surgery is limiting and requires a significant level of dexterity. Moreover, many precision surgery techniques on the ear still requires a bi-manual technique and requires both of the surgeon hands.

### SUMMARY

The present invention solves that problem and provides a hands-free surgical instrument that self-stabilizes in a body orifice, such as an ear canal, as well as being configured for creating an adjustable opening therethrough for one or more medical instruments (such as an endoscope), each of which can be held by the medical device. For example, the medical device can be coupled with and hold a probe that contains both lighting and video imaging hardware, that allows the user to view the surgical site within the ear without having to hold the probe in place. In that manner, the present invention allows both of the user's hands to remain free, while providing both endoscopic visualization and access to traditional bi-manual techniques. Accordingly, the medical devices of the invention provide users with a superior field of view while maintaining hands-free capabilities for surgeons. The medical devices of the invention can be used for procedures that involve any body orifice and find particular use in methods for both routine and complex ear surgeries, such as tympanoplasty (repair of the ear drum), middle ear exploration, cholesteatoma resection, or ossicular chain reconstruction to include stapes surgery.

Aspects of the invention may include a medical device that holds the probe, wherein the device comprises a platform, a probe holder affixed to the platform, wherein the probe holder is configured to hold a probe in a vertical direction, a plurality of spatially adjustable members arranged to form an opening therebetween such that a probe, when situated in the probe holder, extends through the opening and beyond the plurality of spatially adjustable members, and a stabilizer coupled to the platform and positioned behind the plurality of spatially adjustable members and configured to stabilize secure the platform.

The device's platform may secure the probe holder, spatially adjustable members, and stabilizer together and may comprise rotatable knobs or adjustable sliders that allow for adjustment of the device. For example, the platform may comprise of a lead screw adjusted by a rotatable knob, the screw controlling the opening between the spatially adjustable members. The probe holder of the medical device may comprise a laterally adjustable slider to allow for varying distances from the cheek to the ear canal based on subject-specific characteristics. Advantageously, once the rotatable knobs and/or adjustable sliders are set, the device holds the probe in place without further input from the user.

The device's probe holder may comprise a gimbal assembly that allows for adjustment of the angle of entry of the probe along the x and y axes. The probe holder may also comprise cantilever structures that allow the probe to be secured based on friction with the stabilizer. This is advantageous, because the gimbal and cantilever assembly prevent pressure on the body of the probe while maintaining the probe in place. The assembly also allows for a lighter-weight assembly, which is advantageous for both transport and assembly of the device and for placement of the device on the face of the patient for viewing of the ear.

The device's spatially adjustable members may be configured to move by expanding and contracting around the opening. The stabilizer of the device may include a distal portion that extends below the platform. The stabilizer may include a proximal portion that extends above the platform. The distal and/or proximal portions of the stabilizer may be weighted to stabilize the probe. The distal portion of the spatially adjustable members may form a circular or semi-circular shape. Advantageously, the stabilizer may also comprise a ball joint attached to a cheek rest that lies on the cheek bone of the subject, and which is adjustable with a rotatable knob.

The spatially adjustable members may be arranged at an angle for viewing of the ear. For example, the spatially adjustable members may be at a 93° angle from the x-axis for optimal opening of the ear canal of the subject. The spatially adjustable members may be in certain embodiments be a speculum comprising two arms, wherein the two arms when separated from one another apply pressure to the walls of the ear

Aspects of the invention provide a medical device and a probe configured to be situated in the probe holder, the probe comprising a distal end facing the downward end of the probe along the vertical direction when situated in the probe holder and a proximal end facing the upward end of the probe along the vertical direction when mounted. In certain preferred embodiments, the probe may include a video camera rotatably mounted to the distal end of the probe. The camera may be rotatable 180° along the central axis of the probe.

The probe may comprise a housing unit that can house a light emitting diode, a heat sink for that diode, and optical fibers that extend to the distal end of the probe to illuminate the field captured by the video camera, and a data wire that extends to both the proximal and distal ends of the probe. The housing unit may be arranged at the proximal end of the probe, where the probe would be held in place by the probe holder of the device. This is advantageous because heat generated from the diode is kept away from the sensitive ear of the patient being viewed while providing light to the ear.

The proximal end of the probe may comprise a plurality of optical fibers that illuminate the field captured by the video camera, wherein the fibers are arranged in a bias configuration. The camera's lens may lie in the center of the probe or on the opposing end of the bias where the fibers sit. A biased configuration is advantageous because the configuration allows the user to provide light from only one side of the probe, for example only along the wall of the ear or only away from the wall of the ear.

The probe may further comprise a wire that passes from the proximal end of the probe through past the distal end of the probe for communication of the camera's transmissions. The probe may be sheathed in a suitable material. The spatially adjustable members may be in certain embodiments be a speculum comprising two arms, wherein the two arms when separated from one another apply pressure to the walls of the ear; the probe may be inserted through the speculum to insert the camera and lighting into the orifice onto which the device has been mounted. The invention may include the device, the probe, and a user interface comprising a display, the user interface configured to be operably connected to the proximal end of the probe and display video from the camera mounted to the probe.

The user interface may be configured to control the rotation of the camera, or the brightness of the light emitted by the probe. The user interface may comprise a port for the wire that passes through the proximal end of the probe through to the distal end of the probe, thereby operably connecting the user interface to the probe. The user interface may be mounted on external poles, such as IV poles, or on other medical devices that may be found within an operating theater, including on a tabletop, on a stacking unit, or on a pole or mounting arm. The user interface may include holes on the reverse for VESA mounting. The user interface may include sockets for USB for external storage media, power sockets for connecting to external power sources, and display sockets for connecting external display monitors.

Aspects of the invention may include a method of viewing the ear during ear surgery, the method comprising mounting the probe within the medical device, stabilizing the device on the subject, and inserting and adjusting a speculum to lower the probe into the ear canal, and viewing the surgical site using the probe. When mounting the probe, the probe may be held in place by the probe holder of the device by only friction from the cantilever structures of the probe holder. The device may include rotatable knobs that the user manipulates to facilitate insertion of the speculum and probe into the ear canal, other knobs that rotate the probe along the horizontal and vertical axes, allowing for alignment with the ear canal.

The probe may comprise a video camera rotatably mounted to the distal end of the probe, wherein the step of viewing the surgical site using the probe comprises viewing video from the camera. In aspects of the invention, the user may rotate the camera to view the surgical site without rotating the probe itself.

The device and probe may be operably connected to a user interface comprising a display, the user interface configured is operably connected to the proximal end of the probe and configured to display video from the video camera mounted to the probe, wherein the step of viewing the surgical site comprises viewing video from the video camera on the display of the user interface. The user interface may be configured to control the rotation of the camera and viewing the surgical site may use rotation of the camera to view the surgical site using the user interface. In further embodiments, the step of viewing the surgical site may comprise mounting a speculum comprising two arms to the medical device, wherein when the two arms of the speculum are separated from one another they apply pressure to the walls of the ear, lowering the speculum downwards along the vertical direction towards the into the ear canal, and separating the two arms of the speculum.

### BRIEF DESCRIPTION OF THE DRAWINGS

FIG. 1 shows a side view of the device and probe of aspects of the invention.

FIG. 2 shows a top view of the device of aspects of the invention.

FIG. 3 shows a rotated side view of the probe of aspects of the invention.

FIG. 4 shows a side view of the device of aspects of the invention.

FIG. 5 shows a cross sectional view of the device and probe of aspects of the invention.

FIG. 6 shows a bottom view of the distal end of a spatially adjustable member of aspects of the invention.

FIG. 7 shows an alternative bottom view of the distal end of a spatially adjustable member of aspects of the invention.

FIG. 8 shows a plurality of the spatially adjustable members of the device of aspects of the invention.

FIG. 9 shows a plurality of open spatially adjustable members of aspects of the invention.

FIG. 10 shows an anterior view of a spatially adjustable member of aspects of the invention.

FIG. 11 shows a side view of a spatially adjustable member of aspects of the invention.

FIG. 12 shows the distal end of the probe of aspects of the invention.

FIG. 13 shows a cross sectional view of the probe of aspects of the invention.

FIG. 14 shows a rotated anterior and side view of the user interface of aspects of the invention.

FIG. 15 shows a sample design of the screen of the user interface of aspects of the invention.

FIG. 16 shows a rotated side and posterior view of the user interface of aspects of the invention.

FIG. 17 shows a posterior and underside view of the user interface of aspects of the invention.

FIG. 18 illustrates one embodiment of the stabilizer comprising a T-Bar as a cheek rest.

FIG. 19 illustrates one embodiment of the T-Bar arms for attaching accessories to the T-Bar.

FIG. 20 illustrates one embodiment of a head strap of devices of the invention.

FIG. 21 illustrates one embodiment of a head strap which includes an eye mask.

FIG. 22 illustrates one embodiment of a weighted pad as a stabilizer to support the probe holder and stabilizer.

FIG. 23 and FIG. 24 illustrate embodiments of a clip molded to the cable of the probe.

FIG. 25 illustrates one embodiment of the device.

FIG. 26 illustrates one embodiment of devices of the invention.

### DETAILED DESCRIPTION

The present invention provides a self-stabilized medical device. In certain embodiments, the medical devices of the invention are optionally coupled with and hold a probe that contains both lighting and video imaging hardware, that allows a user to view a surgical site within a body orifice (such as an ear) without having to hold the probe in place. The present invention allows both of a user's hands to remain free, providing both endoscopic visualization and access to traditional bi-manual techniques. The medical devices of the invention can be used for procedures that involve any body orifice and find particular use in methods for both routine and complex ear surgeries, such as tympanoplasty (repair of the ear drum), middle ear exploration, cholesteatoma resection, or ossicular chain reconstruction to include stapes surgery.

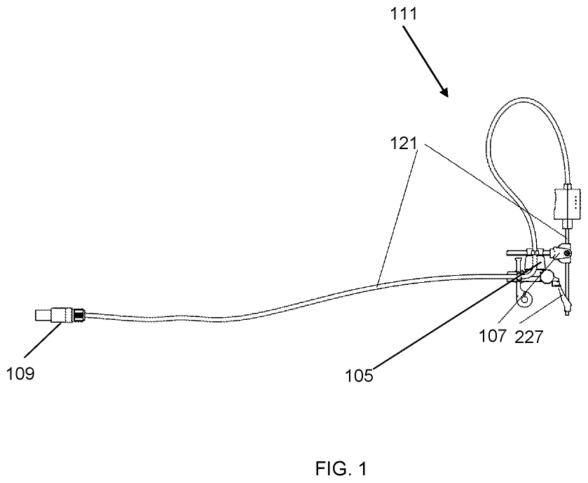

FIG. 1 shows a side view of a platform 105 and a probe 121 of aspects of the medical device 111 of invention. The probe 121 is attached to the platform 105 and ready for insertion in the ear. The proximal end of the probe comprises a housing 109 and a cable to the user interface (not pictured).

The medical device comprises a platform 105. The platform 105 has attached thereto a probe holder 107, a plurality of spatially adjustable members 127, and a stabilizer. The device may be constructed from metal or of suitable engineered plastics or a combination thereof and may comprise materials that can be sufficiently sterilized for surgical theaters. The platform 105 of the device combined with the probe holder, the plurality of spatially adjustable members, and the stabilizer, are able to achieve six separate adjustable measurements for fitting the device onto the cheek and over the ear of a subject. The adjustability of the device is important to the ability of the system to be able to be operated by an operator without keeping a hand on the device, probe, or user interface at all times. This achieves the desired ability of the operator to use bi-manual techniques for ear surgery.

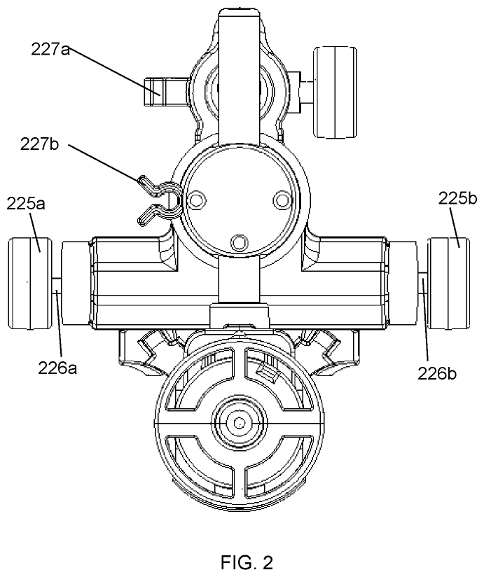

FIG. 2 shows a top view of a device of the invention. The lead screw 226a and 226b allow the adjustment of the opening of speculum blades that hold the ear canal open during surgery The screws are adjusted using rotatable knobs 225a, 225b. The device may include catches 227a, 227b that are used to secure excess length of the probe to ensure that the probe sheath is not entangled during surgery.

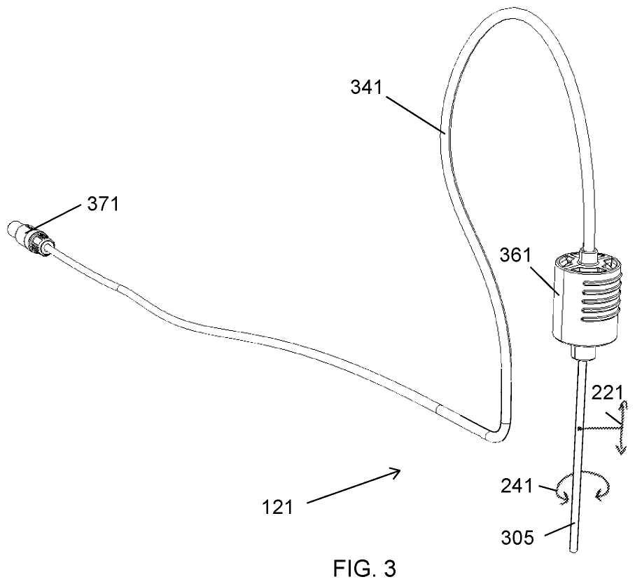

FIG. 3 shows a rotated side view of the device 121 of aspects of the invention. A cable 341 connects the proximal end of the probe 361 to the user interface through an input 371. The probe includes a cylindrical body 221 connecting the LED housing 361 at the proximal end of the probe that is secured by the probe holder (not pictured) to the proximal and distal ends of the probe. The distal end 305 of the probe is inserted into the ear canal. The probe may be rotated around a vertical axis 241 or may be raised and lowered vertically 221.

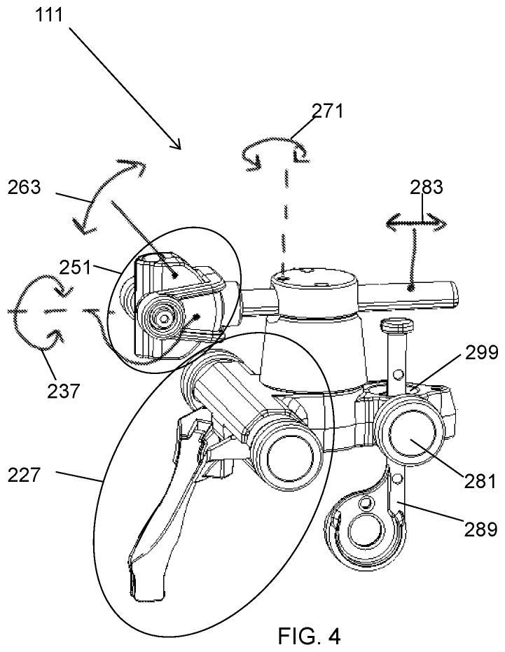

FIG. 4 shows a side view of a medical device 111 of aspects of the invention. The device comprises a probe holder, which comprises a gimbal assembly 251 and secures the probe so that it can be inserted into the ear canal. The angle 263 of the gimbal assembly may be adjusted along the x and z axes to account for the angle of the ear canal of the subject. The gimbal assembly may also be rotated about the horizontal axis 237 to further account for the angle of the ear canal of the subject. The probe is inserted through the adjustable speculum 227, which comprises two arms arranged to form an opening through which the probe is inserted, and from thence into the ear canal. The device includes a stabilizer 289, that may allow the device to be rested on the cheek of the subject. The stabilizer may be secured by a ball joint 299. It may be adjusted by the adjacent knob 281 to adjust the movement of the device. The gimbal assembly 251 further allows for lateral movement 283 of the device, for example once stabilized. Further the gimbal assembly 251 can translate laterally in a swinging motion with the turret 520 (not pictured), through a vertical axis 271.

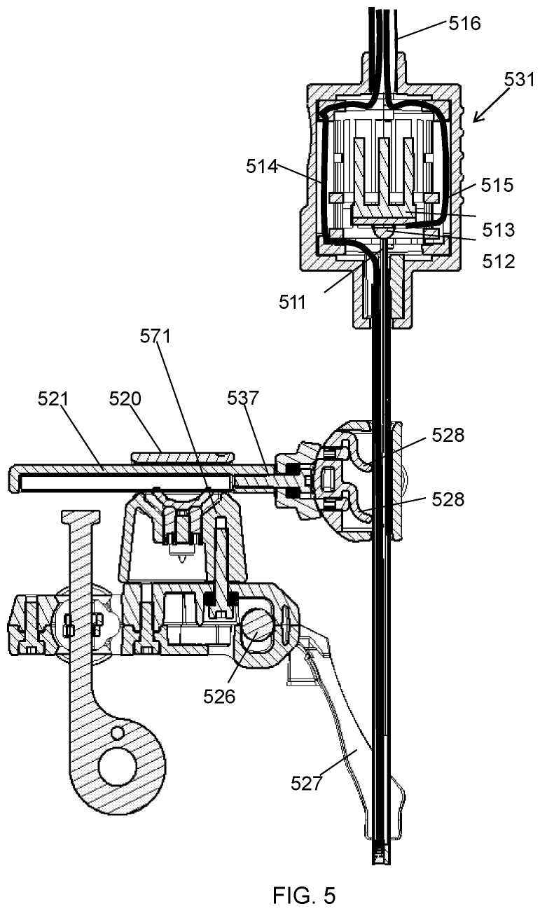

FIG. 5 shows a cross sectional view of a medical device and probe of aspects of the invention. The probe includes, from the proximal to distal end, a sheath 516 that leads to the LED housing 531, in which may be found components optical fibers 511, the LED 512, a heat sink 513, a data wire 514, LED wires 515, from which then leads the probe shaft that proceeds to the distal end. Parts of the device shown include a turret 520 that is part of the platform, slider 521 that is part of the probe holder, a lead screw 526 that is the connection point between the platform and the arms of the speculum 527, and a cantilever structure 528 that is part of the probe holder. Point 571 is the axis about which the gimbal assembly can translate around its axis. Point 537 is the axis about which the probe holder can rotate around the horizontal axis.

The probe holder of the device proceeds through the turret 520 of the platform to secure the probe to the platform, which allows for the lateral adjustment of the probe holder to allow for subject-specific distance, for example between the cheek bone on which the platform stabilizer may rest, and the ear canal into which the probe is inserted. The probe holder secures the probe using the cantilever structures 528 that use friction to keep the probe in place.

The probe holder is designed to fit the probe within it, wherein the outer sheath of the probe will be in contact with the probe holder at the cantilever structures. The probe holder is designed that the housing unit of the probe is positioned proximally, that is toward the user and away from the ear.

The arms of the speculum on the device form an opening at their distal portion. That opening forms a circular shape. In certain preferred embodiments, by turning the lead screw, the operator can move the arms or blades (used interchangeably) about 2 mm from their resting position. The blades are designed with a concave shape, with about a 3 mm distance between the edge of the speculum blade at its proximal point, and the apex of the arc of the concave curve of the blade. This allows an opening of about 10 mm in diameter. Additionally, the curve of the blade is designed to be angled about 93° from the x-axis; this preferentially allows for the safe retraction of the ear canal and for optimal patency of the ear canal.

The blades are inserted closed into the outer ear and then the ear canal. Once in the ear canal, the blades are spatially adjustable and may be opened to provide the space for lowering the probe into the ear canal and then for viewing of the surgical site. Spatial adjustment of the blades refers preferentially to their movement outward from the center of the ear canal. The members are fixed in length and in the diameter of the opening they create.

The stabilizer may comprise a ball joint that couples the stabilizer to the platform. The ball joint allows for rotational movement. The stabilizer may further comprise of a cheek rest that sits on the subject helping to balance and stabilize the device on the subject's head. The stabilizer may be adjustable with a rotatable knob. The knob enables the rest to be extended along the y-axis, adjusting for subject-specific cranial characteristics.

The probe of the device is configured to be situated in the probe holder, the probe comprising a distal end facing the downward end of the probe along the vertical direction when situated in the probe holder and a proximal end facing the upward end of the probe along the vertical direction when mounted. The probe may comprise at least a shaft or sheath, optical fibers, a camera lens, an image sensor, a data wire, a light emitting diode, and a heat sink for that diode. The probe preferentially comprises a housing unit for the light emitting diode, the heat sink for that diode, the optical fibers that extend to the distal end of the probe to illuminate the field captured by the video camera, and the data wire that extends to both the proximal and distal ends of the probe.

The shaft or sheath of the probe may be manufactured from any inert material appropriate for a surgical environment. The shaft or sheath of the probe is preferentially not a conductive material and may be secured to the platform with catches when there is excess length to reduce risk of entanglement. The shaft or sheath may be created in one or a plurality of sections, each of which may be of differing or the same materials; manufacture of the sheath is well known to persons having ordinary skill in the art.

The housing unit may be manufactured from any material appropriate for a surgical environment that can also suitably hold the required components within it. The light emitting diode may be a semiconductor light source that emits light when current flows through it, wherein electrons in the semiconductor recombine with electron holes, releasing energy in the form of photons. The light emitting diode is known to persons having ordinary skill in the art; it also may require a heat sink that may also be found in the housing unit. The heat sink is a passive mechanism for excess heat energy to flow away from the source and to a medium that helps keep the temperature of the source of the heat energy low. The heat sink may be manufactured from any suitable material and is known to persons having ordinary skill in the art.

The distal end of the probe may comprise a video camera rotatably mounted. The video camera may be any manufacturer's camera of sufficient size and with the ability to view with sufficient detail the surgical site. The camera may be rotated 180° along the central axis of the probe. The video camera may be able to take real time still and video images of the surgical site. The video camera may be manufactured according to the techniques known to persons having ordinary skill in the art.

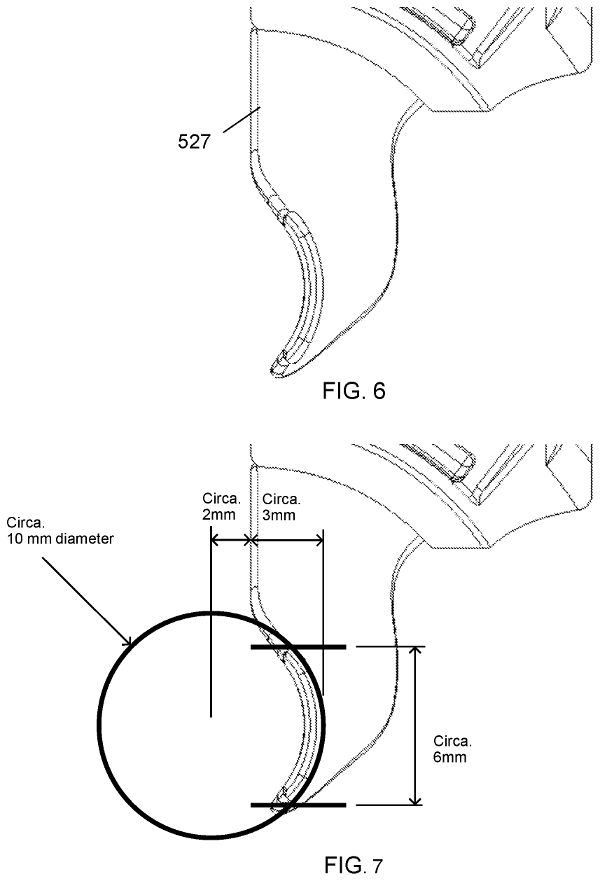

FIG. 6 shows a bottom view of the distal end of an adjustable speculum blade 527, a spatially adjustable member of a device of the invention. This side view demonstrates the curvature of the speculum blade designed for safe inserting into and opening of the ear canal.

FIG. 7 shows an alternative bottom view of the distal end of an adjustable speculum blade. The view demonstrates the exemplary measurements of each of the curvatures of the blades and the opening that the speculum blades would create. Advantageously, because the blades are adjustable, they may be used for a range of ear canal sizes.

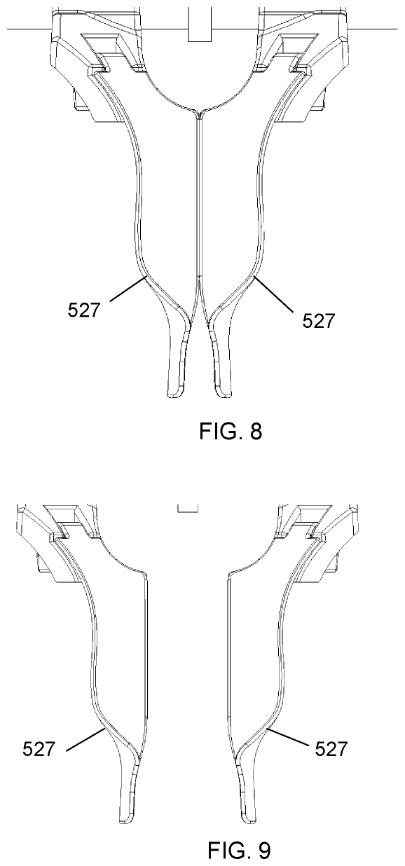

FIG. 8 shows two of the adjustable speculum blades 527 when closed. The view demonstrates the device as it would be ready for insertion into the ear canal of a subject.

FIG. 9 shows two of the adjustable speculum blades 527 when open. This view demonstrates the device as it would be ready for insertion of the probe. Each of the blades is in contact with the walls of the ear canal.

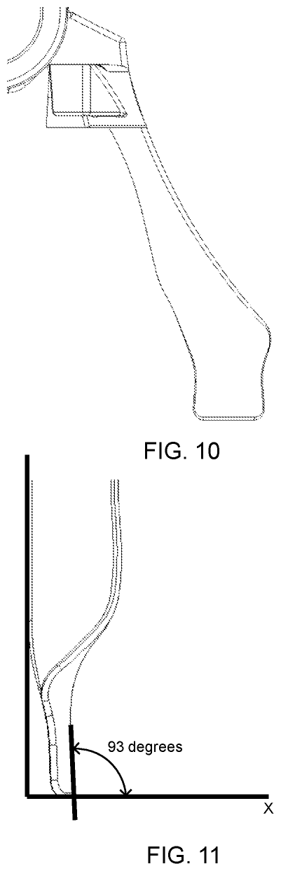

FIG. 10 shows an anterior view of the adjustable speculum blades. The blades are shaped to be nonintrusive during ear surgery. For example, they have been cut down with the lowest point facing the surgeon, thus allowing maximum access for articulating tools in the ear canal, while also retracting the ear and ear canal to provide access for the probe and surgical tools.

FIG. 11 shows a side view of an adjustable speculum blade. The view demonstrates an exemplary angle at which the blade is designed, where the speculum will create the opening to access the ear canal. A feature of the blades is that they are tapered outwards. When in use, the taper applies a force on the tissues of the ear which prevent the speculum from slipping out of position or out of the ear canal.

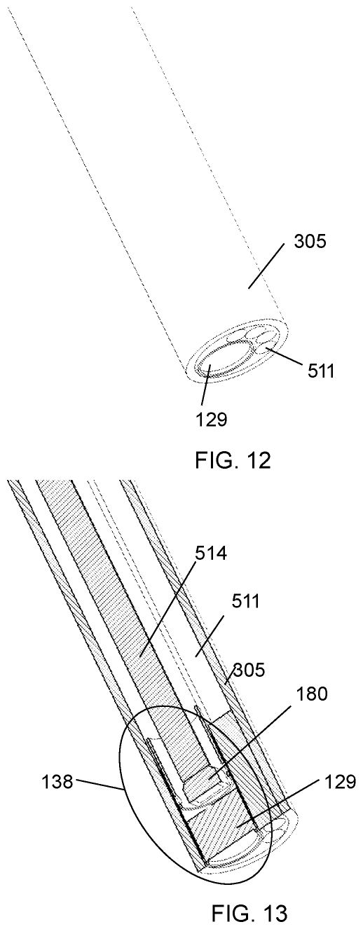

FIG. 12 shows the distal end of the probe of aspects of the invention. At the distal end of the probe shaft 305 may be found the lens 129 of the video camera. There are also the ends of optical fibers 511 that illuminate the ear canal; the fibers proceed from the LED that is contained within the housing unit on the probe. The ends of a plurality of optical fibers are shown in the bias configuration which is a preferential embodiment of the invention. Advantageously, this allows for the rotation of the probe about the horizontal plane to cause the illumination emitting from the probe to light the ear most optimally.

FIG. 13 shows a cross sectional view of the probe of aspects of the invention. Within the probe shaft 305 are housed optical fibers 511, the data wire 514, and a video camera 138; the video camera further comprises an image sensor 180 and the lens 129, for example a CMOS image sensor.

The user interface comprises a display and is operably connected to the proximal end of the probe and display video from the camera mounted to the probe. The user interface may control the rotation of the video camera and the brightness of the LED of the probe. The user interface may further comprise a port for the wire that passes from the proximal end of the probe through past the distal end of the probe, thereby operably connecting the user interface to the probe. The user interface may further comprise data ports that enable connection to external displays. The user interface may be mountable on external poles, mounting arms, or other medical equipment to facilitate use in surgical environments.

The user interface may comprise parts including a housing unit, ports for connectivity to power, data inputs and outputs, a screen, and manual switches as described herein and shown in the figures. The user interface may be manufactured according to techniques known to persons having ordinary skill in the art. The housing unit and screen of the user interface may be manufactured from any materials suitable for a surgical environment. The user interface may optionally be covered with a disposable plastic sheet or other material suitable for a surgical environment for cleaning between subjects.

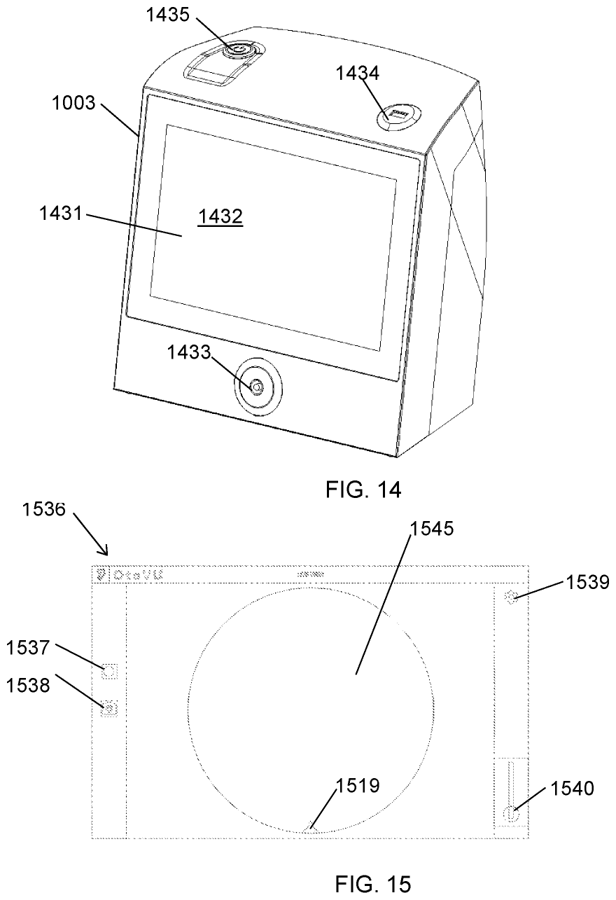

FIG. 14 shows a rotated anterior and side view of a user interface. The user interface is housed in a camera control unit 1003. The user interface is on a display 1431 and is a touch sensitive screen 1432. The socket 1433 is the port into which the proximal end of the probe 121 is inserted. The USB socket 1434 allows for interface with external computer systems. The power button 1435 turns on the user interface.

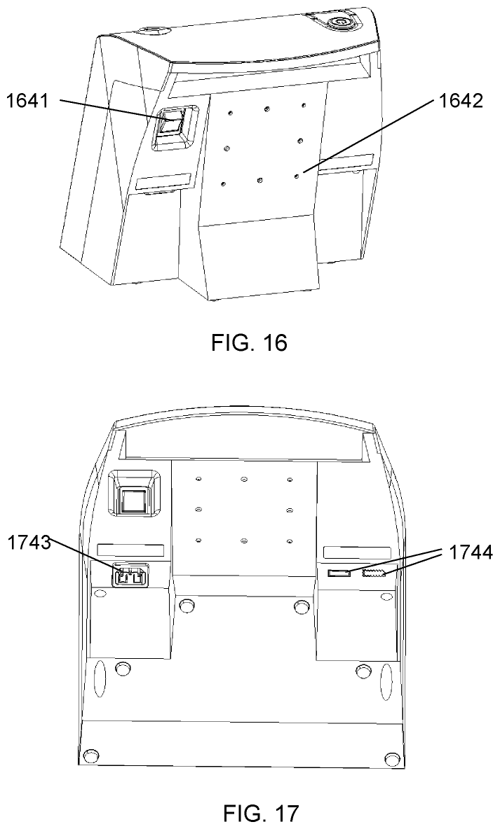

FIG. 15 shows a sample design of the screen 1536 of the user interface. The user interface may include a color balancing button 1537. It may include a button to control the recording of video and pictures 1538. The interface may include an orientation indicator 1519 of which direction the camera is currently displaying. The interface may include a settings button 1539. The interface may include a slide 1540 that controls the brightness of the LED. The circular image 1545 transmitted by the camera may be shown prominently on the display FIG. 16 shows a rotated side and posterior view of the user interface. The interface's control unit may include a power isolation switch 1641. It also may include holes for a mount, for example a VESA mounting adapter 1642.

FIG. 17 shows a rotated posterior and underside view of the user interface. The control unit may include a power socket 1743 for electrical power cables to be inserted into the device. It may have a plurality of display sockets 1744 that allow extension of the display to external monitors.

The medical devices of the invention can be coupled with various different medical instruments and probes for use in various medical procedures in different body orifices. In certain embodiments, the invention provides methods of viewing within an ear during ear surgery. Such methods may involve mounting a probe to the medical device and attaching the probe to the user interface, then lowering the probe downwards along the vertical direction towards the into the ear canal, and then viewing the surgical site using the probe.

The step of mounting the probe to the medical device may comprise holding the probe in place via friction at the cantilever structures of the probe holder of the device. Lowering the probe may require that the device be adjusted laterally, that the device be rotated about the horizontal axis, that the angle of the entry of probe holder be adjusted, that the probe be rotated about the vertical axis, that the stabilizer be adjusted for depth, and finally that the spatially adjustable members be opened for insertion of the probe. The adjustments may comprise six adjustments for fitting the system on a subject for bi-manual surgical techniques.

Because the device may comprise a plurality of rotatable knobs, the step of viewing the surgical site may be comprise opening the spatially adjustable blades to enable the lowering of the probe into the ear canal. Furthermore, the step of viewing the surgical site may comprise adjusting the device with the rotatable knobs so the probe is appropriately inserted into the ear canal of the subject.

In certain preferred embodiments, the probe comprises a video camera rotatably mounted to the distal end of the probe, and wherein the step of viewing the surgical site using the probe comprises viewing video from the camera. The step of viewing the surgical site may comprise rotating the camera to view the surgical site and/or adjusting the brightness of the illumination from the probe.

The probe may operably be connected to a user interface comprising a display, the user interface configured to be operably connected to the proximal end of the probe and to display video from the video camera mounted to the probe. The step of viewing the surgical site may comprise viewing video from the video camera on the display of the user interface.

Advantageously, the user interface may control the rotation of the camera and the step of viewing the surgical site may comprise rotating the camera to view the surgical site using the user interface. The user interface may include an orientation marker to denote which position the probe is occupying in the ear canal.

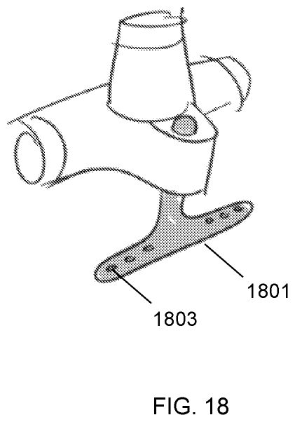

FIG. 18 illustrates one embodiment of the stabilizer comprising a T-Bar 1801 as a cheek rest. In some embodiments, the cheek rest may be a T-Bar 1801 substantially shaped as a “T” that includes a wide platform, arms, or wings extending from the base of the T-Bar. The wings or arms may extend oppositely and laterally outwards from the base of the T-Bar. The base of the T-Bar may be connected to the stabilizer, with the platform, wings or arms of the “T” extending to rest on the patient's head.

In this way, the platform, arms, or wings of the T-Bar may be used for stabilizing the probe stabilizer on the patient's head. For example, the T-Bar may be configured to add stability at the rear of the stabilizer, width to the stabilizer, and/or both.

As noted above, the stabilizer may comprise a ball joint that couples the stabilizer to the platform. The ball joint allows for rotational movement. The T-Bar may be attached to the stabilizer via a ball joint such that the T-Bar is adjustable with a rotatable knob. The knob enables the T-Bar to be extended along the y-axis, adjusting for subject-specific cranial characteristics.

The platform of the T bar may include one or more holes or openings 1803 to allow for suturing. For example, the openings may be configured such that suturing may occur through the openings.

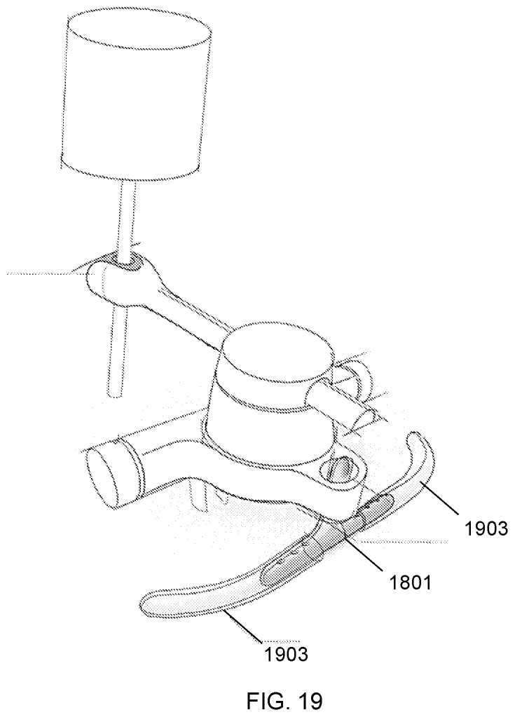

FIG. 19 illustrates one embodiment of the T-Bar 1801 wherein the T-Bar comprises arms for attaching accessories to the T-Bar. In embodiments, the T-Bar may comprise an attachment mechanism for attaching various accessories to the T-Bar arms. The attachment mechanism may be a slot or clip, for example. The T-Bar may be configured for slidably attachable accessories, for example flexible arms 1903, to slide onto the arms of the T-Bar. The accessories may be separately attachable to the arms of the T-Bar and removable to provide specific stabilization. For example, in embodiments, the accessories or flexible arms may be identical. In other embodiments, the accessories or flexible arms may be differently shaped and configured for adjusting for subject-specific cranial characteristics. The accessories or flexible arms may be calibrated as needed. As noted above, the T-Bar may comprise openings for suturing, the area of which may be extended using flexible arms. Thus, in embodiments, the T-Bar cheek rest can support accessories attachable to the T-Bar to meet a variety of specific needs.

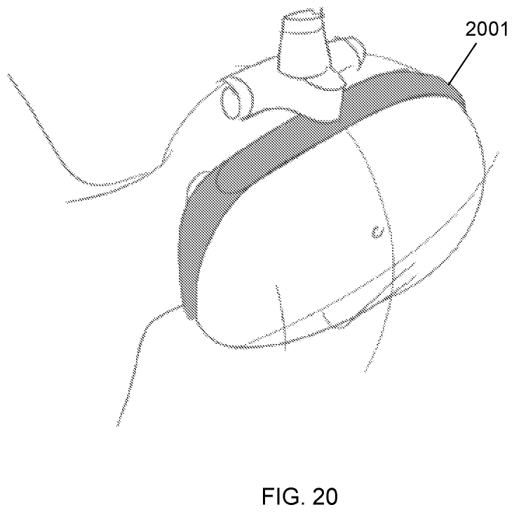

FIG. 20 illustrates one embodiment of a head strap 2001 of devices of the invention to provide stability to the platform. In embodiments, the head strap is a flexible material that is attached to the probe stabilizer to provide stability to the platform. For example, the head strap may be made of a molded silicone/TPE. The head strap may be wrapped partially or fully around the patient's head, for example the head strap may be wrapped between the top of the head and under the chin. Alternatively, in some embodiments, the head strap may be wrapped at other positions on the head, for example from the front of the head to the back of the head. In some embodiments, the head strap may be attached to the T-Bar, for example by connecting to the arms of the T-Bar. The head strap may include pockets in which to slide the arms of the T-Bar so as to connect the head strap to the T-Bar.

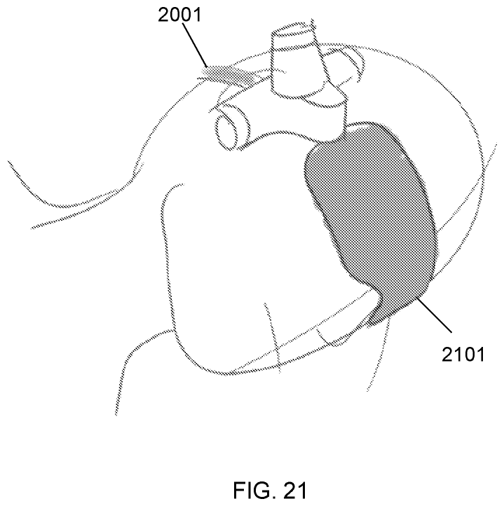

FIG. 21 illustrates one embodiment of a head strap 2001 which includes an eye mask. 2101. The head strap may be wrapped front to back on the patient's head. The eye mask may be used to protect the patient's eyes during surgery and to provide a comfortable fitting of the strap.

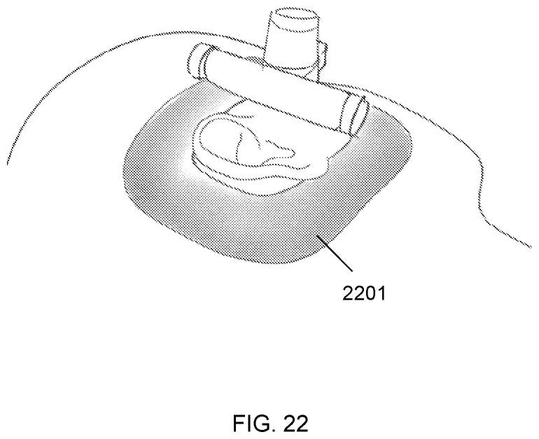

FIG. 22 illustrates one embodiment of a weighted pad 2201 as a stabilizer to support the probe holder and stabilizer. In embodiments, the weighted pad is made of a flexible material. The material may be a flexible polymer. The weight of the weighted pad may stabilize the probe without the need for a strap or suture. In combination with the stabilizer and probe the weighted pad may act to bring the center of gravity of the combined device within the volume enclosed by the speculum arms, blade, or leaves as part of the probe stabilizer. In some embodiments, the weighted pad may be used in combination with the head strap or T-Bar for stabilization.

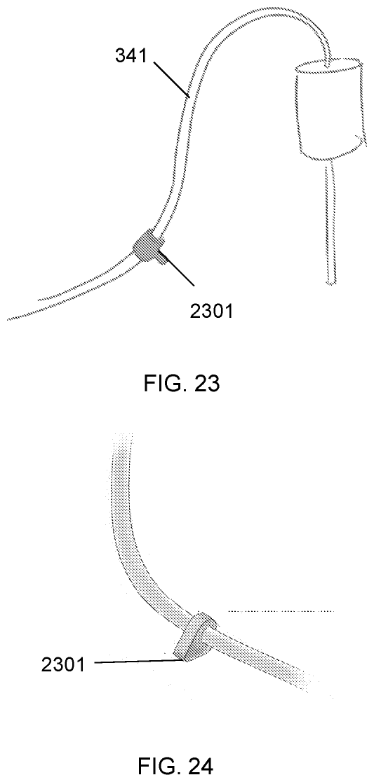

FIG. 23 and FIG. 24 illustrate embodiments of a clip 2301 molded to the cable 341 of the probe. As noted above, the device may include catches that are used to secure excess length of the probe to ensure that the probe sheath is not entangled during surgery. The clip 2301 allows the probe cable to be attached to the probe holder. The cable clip may be attached to the probe holder via a snap or click mechanism, for example by attaching to the rear end of the slider. This centers the cable and allows for movement together with any forward/backward probe or scope adjustments. In this way, the cable clip may provide a rotational alignment of the cable and probe relative to the probe supporter. This ensures the probe is oriented in an upright position for visualizing the surgery.

The cable clip may comprise a knob or peg for attaching to the device The clip may be made of a molded color plastic to visually offset the clip from the cable or other aspects of the device. In some embodiments, the cable clip is slidable along the cable such that the cable can pass through the cable clip. In other embodiments, the cable clip is positioned at a fixed point on the cable, for example, the cable clip may be molded directly to the cable or directly to the slider.

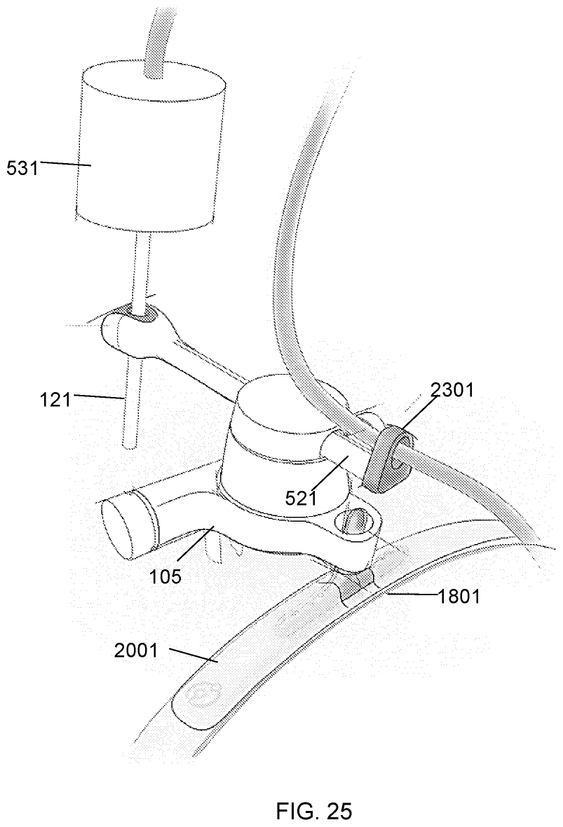

FIG. 25 illustrates one embodiment of the device. As noted above, the probe 121 is secured in the probe holder and may include a sheath leading to the LED housing 531 at a proximal end of the probe. The cable clip may be attached to the rear end of the slider 521 of the probe holder to center the cable and allow for movement together with the movement of adjustments to the probe 121. Thus, rotational alignment of the cable and probe relative to the probe holder may be achieved, and ensures that the probe is oriented in an upright position for visualizing the surgery. As illustrated in the embodiment, the stabilizer may comprise a T-Bar 1801 as a cheek rest, wherein the T-Bar includes arms, wings, or a platform to which accessories, such as a head strap 2001, may be attached.

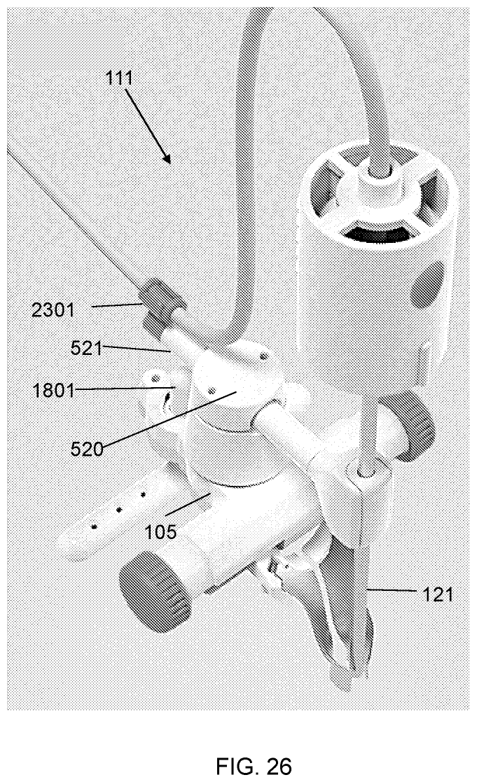

FIG. 26 illustrates one embodiment of devices 111 of the invention. The stabilizer may comprise a T-Bar 1801 as a cheek rest attached to the platform 105. The T-Bar may include openings for suturing. As described above, the probe holder of the device proceeds through the turret 520 of the platform to secure the probe to the platform, which allows for the lateral adjustment of the probe holder to allow for subject-specific distance, for example between the cheek bone on which the platform stabilizer may rest, and the ear canal into which the probe is inserted. The probe holder secures the probe 121 using the cantilever structures that use friction to keep the probe in place. The cable clip 2301 may be attached to the rear end of the slider 521 of the probe holder to center the cable and allow for movement together with the movement of adjustments to the probe 121.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

We claim:

1. A medical device comprising:
a platform;
a probe holder affixed to the platform, wherein the probe holder is configured to hold a probe in a vertical direction;
a plurality of spatially adjustable members arranged to form an opening therebetween such that a probe, when situated in the probe holder, extends through the opening and beyond the plurality of spatially adjustable members; and
a stabilizer coupled to the platform and positioned behind the plurality of spatially adjustable members and configured to secure the platform.

2. The medical device of claim 1, wherein the platform comprises a turret that secures the stabilizer.

3. The medical device of claim 1, wherein the platform is further comprised of a lead screw adjusted by a rotatable knob, the screw controlling the opening between the spatially adjustable members so that a probe can be inserted through the opening.

4. The medical device of claim 1, wherein the probe holder is further comprised of a gimbal assembly that allows for adjustment of the angle of entry of the probe along the x and y axes.

5. The medical device of claim 1, wherein the probe holder is further comprised of cantilever structures that allow a probe to be secured based on friction with the stabilizer.

6. The medical device of claim 1, wherein the probe holder is further comprised of a laterally adjustable slider to allow for varying distances from the cheek to the ear canal.

7. The medical device of claim 1, wherein the opening formed by the distal portion of the spatially adjustable members forms a circular or semi-circular shape.

8. The medical device of claim 1, wherein the distal portion of the spatially adjustable members is angled 93° from the x-axis for optimal opening of the ear canal of the subject.

9. The medical device of claim 1, wherein the stabilizer is further comprised of a ball joint attached to a cheek rest that lies on the cheek bone of the subject, and which is adjustable with a rotatable knob.

10. A kit comprising:
the medical device according to claim 1; and
a probe configured to be situated in the probe holder, the probe comprising a distal end facing the downward end of the probe along the vertical direction when situated in the probe holder and a proximal end facing the upward end of the probe along the vertical direction when mounted.

11. The kit of claim 10, wherein the probe comprises a video camera rotatably mounted to the distal end of the probe.

12. The kit of claim 10, wherein the video camera is rotatable 180° along the central axis of the probe.

13. The kit of claim 10, wherein the probe comprises a housing unit that can house a light emitting diode, a heat sink for that diode, optical fibers that extend to the distal end of the probe to illuminate the field captured by the video camera, and a data wire that extends to both the proximal and distal ends of the probe.

14. The kit of claim 10, wherein the proximal end of the probe comprises a plurality of optical fibers that illuminate the field captured by the video camera, wherein the fibers are arranged in a bias configuration.

15. The kit of claim 10, wherein the probe is further comprised of a wire that passes from the proximal end of the probe through past the distal end of the probe for communication of the camera's transmissions.

16. The kit of claim 10, further comprising a speculum comprising two arms, wherein the two arms when separated from one another apply pressure to the walls of the ear.

17. A medical system comprising:
the medical device according to claim 1;
a probe configured to be situated in the probe holder, the probe comprising a distal end facing the downward end of the probe along the vertical direction when situated in the probe holder and a proximal end facing the upward end of the probe along the vertical direction when mounted; and
a user interface comprising a display, the user interface configured to be operably connected to the proximal end of the probe and display video from the camera mounted to the probe.

18. The system of claim 17, wherein the user interface is configured to control the rotation of the video camera.

19. The system of claim 17, wherein the user interface is configured to control the brightness of the LED of the probe.

20. The system of claim 17, wherein the user interface is further comprised of a port for the wire that passes from the proximal end of the probe through past the distal end of the probe, thereby operably connecting the user interface to the probe.

21. The system of claim 17, wherein the user interface is further comprised of data ports that enable connection to external displays.

22. The system of claim 17, wherein the user interface is mountable on external poles, mounting arms, or other medical equipment to facilitate use in surgical environments.

23. The system of claim 17, further comprising a speculum comprising two arms, wherein the two arms when separated from one another apply pressure to the walls of the ear.

24. A method of viewing the ear during ear surgery, the method comprising:
mounting a probe to a medical device for stabilizing the probe, the medical device comprising:
a platform;
a probe holder affixed to the platform, wherein the probe holder holds the mounted probe in a vertical direction;
a plurality of spatially adjustable members arranged to form an opening therebetween such that the mounted probe extends through the opening and beyond the plurality of spatially adjustable members; and
a stabilizer coupled to the platform and positioned behind the plurality of spatially adjustable members and configured to secure the mounted probe,
lowering the probe downwards along the vertical direction towards the into the ear canal; and
viewing the surgical site using the probe.

25. The method of claim 24, wherein the step of mounting the probe to the medical device comprises holding the probe in place via friction at the cantilever structures of the probe holder of the device.

26. The method of claim 24, wherein the medical device is further comprised of a plurality of rotatable knobs, and wherein the step of viewing the surgical site comprises opening the spatially adjustable members to enable the lowering of the probe into the ear canal.

27. The method of claim 24, wherein the medical device is further comprised of a plurality of rotatable knobs, and wherein the step of viewing the surgical site comprises adjusting the device with the rotatable knobs so the probe is appropriately inserted into the ear canal of the subject.

28. The method of claim 24, wherein the probe comprises a video camera rotatably mounted to the distal end of the probe, and wherein the step of viewing the surgical site using the probe comprises viewing video from the camera.

29. The method of claim 24, wherein the probe is operably connected to a user interface comprising a display, the user interface configured to be operably connected to the proximal end of the probe and to display video from the video camera mounted to the probe, and wherein the step of viewing the surgical site comprises viewing video from the video camera on the display of the user interface.

30. The method of claim 24, wherein the user interface is configured to control the rotation of the camera imaging and wherein the step of viewing the surgical site comprises rotating the camera imaging to view the surgical site using the user interface.

31. The method of claim 24, further comprising the steps of mounting a speculum comprising two arms to the medical device, wherein when the two arms of the speculum are separated from one another they apply pressure to the walls of the ear, lowering the speculum downwards along the vertical direction towards the into the ear canal, and separating the two arms of the speculum.
